(19) United States
(12) Patent Application Publication
(10) Pub. No.: US 2016/0115469 A1
(43) Pub. Date: Apr. 28, 2016

(54) DEVICE FOR ELECTRICALLY DISINTEGRATING CELL CLUSTERS

(71) Applicants: HUGO VOGELSANG MASCHINENBAU GMBH, Essen (DE); PROMETHEUS GMBH & CO. KG, Löningen-Bunnen (DE)

(72) Inventor: Paul Flerlage, Löningen-Bunnen (DE)

(21) Appl. No.: 14/896,216

(22) PCT Filed: Jun. 4, 2014
(86) PCT No.: PCT/EP2014/061548

§ 371 Date: Dec. 4, 2015

(30) Foreign Application Priority Data
Jun. 4, 2013 (DE) 20 2013 005 125.6

(51) Int. Cl. C12N 13/00

(52) U.S. Cl. CPC C12N 13/00

(57) ABSTRACT

A device (1) for electrically disintegrating cell clusters, comprising: an electrode unit (11) which has an electrode head (13) and an electrode body (15); a chamber (5) within which the electrode body (15) is arranged, wherein the chamber (5) has a wall (19) which is electrically conductive in some sections or completely and which is electrically insulated from the electrode body (15), and wherein the chamber (5) has an inlet (7) for receiving fluid containing cell clusters; a high-voltage source (56) which is arranged in the electrode head (13) and which is designed to produce an electric field by applying a voltage between the electrode body (15) and the wall (19), and an electronic control unit (29), which interacts with the high-voltage source in order to change the electric field.

According to the invention, the electronic control unit has means for determining the resonance frequency of the high-voltage source (56).

The present invention relates to a device for electrically disintegrating cell clusters, comprising: an electrode unit which has an electrode head and an electrode body; a chamber within which the electrode body is arranged, wherein the chamber has a wall which is electrically conductive in some sections or completely and which is electrically insulated from the electrode body, and wherein the chamber has an inlet for receiving fluid containing cell clusters; a high-voltage source which is arranged in the electrode head and which is designed to produce an electric field by applying a voltage between the electrode body and the wall, and an electronic control unit which interacts with the high-voltage source in order to change the electric field.

Such a device is known from utility model DE 20 2011 004 177 U1, which is held by the present applicant. Such devices are used in different fields, mainly for treating mixtures of fluid with organic material, in particular mixtures containing cells and/or cell clusters, in biogas plants and sewage plants. The aim is to foster the production of biogas by disintegration of cell clusters, for example in biogas plants, because cracking cell clusters will favour the reaction of starting materials to produce digester gas. The expression disintegration is generally understood to mean the comminution of cells or cell clusters under the action of external forces.

Other known disintegration methods are thermal disintegration, ultrasound disintegration, chemical disintegration and mechanical disintegration.

Electrical disintegration is based on the functional principle of exposing cell clusters to an electric field produced between two electrodes. Due to the effect of the electric field on the cells and cell clusters, charge transfers occur at the cell membranes. Known systems for electrical disintegration exploit the fact that cells and cell clusters move inside the chamber in which the electric field is produced. The movement of cells and cell clusters causes changes in the strength of the field that acts locally on their respective cell membrane. Due to this continuous change, the cell membrane and/or the cell cluster is exposed to shear forces and vibrations, which results in its destabilisation.

If excitation is sufficiently strong, the cell cluster is loosened or broken up. If the influence exerted is even stronger, the cell membranes collapse. The latter process is known under the term electroporation. The effect of such disintegration is that nutrient availability for fermenting bacteria is significantly increased. This effect is advantageously exploited in biogas plants to increase the gas yield and to put the deposited substrates to better use. Systems which use the principle of electrical disintegration and the method of electrical disintegration are superior to the alternative disintegration methods in respect of investment expense, energy input and the amount of equipment involved.

In the DE 20 2011 004 177 U1 utility model kind referred to above, it is proposed that the efficiency of disintegration be enhanced by a control unit cooperating with the high-voltage source in order to alter the electric field, the control unit being adapted to change the voltage between the electrode and the wall. It has been found that varying the electric field results in a significant increase in the efficiency of disintegration. The efficiency of disintegration and hence also its possible increase by means of the device proposed in DE 20 2011 004 177 U1 is dependent, however, on the mixtures of fluid and organic material that are used to produce gas, for example on whether renewable raw materials or abattoir waste are used, and on the extent to which the organic material has already been disintegrated. There is therefore a need to optimise the efficiency of disintegration in such a way that the device for electrically disintegrating cell clusters can be rapidly adapted to changing ambient conditions, in particular so that it can be rapidly adapted to changed properties of the organic material, namely in such a way that, for a given input voltage for the device, as strong an electric field as possible is available for disintegration in the chamber.

However, it has been found with prior art devices that, due to their constructional design, for example due to earthing of the chamber, it is not possible to measure the electric field inside the chamber directly without further ado, which meant a reliance on plant operating parameters that were predetermined by calibration for the respective fluids to be expected.

The object of the invention is therefore to specify a device for electrical disintegration which can be rapidly adapted to changing ambient conditions, and which can be adapted, more specifically, to changed properties of the organic material.

The invention solves the problem it addresses with a device of the kind referred to at the outset, in which the electronic control unit has means for determining the resonance frequency of the high-voltage source. The invention is based on the realisation that the high-voltage source forms a resonant circuit with the electrode body and the chamber wall. As soon as there is a change in the temperature, viscosity, pressure or volumetric flow rate of the fluid in the chamber, there is also a change in permittivity in the chamber. This, in turn, affects the resonance frequency of the resonant circuit, in accordance with generally known principles of physics. Given that optimal generation of a field is also assured at or at least near the resonance frequency, determining said frequency has been found to be an appropriate measure for responding to changing conditions in the chamber.

In one preferred development of the invention, the high-voltage source has a high-voltage coil and a measuring coil, wherein the measuring coil is connected to the means for determining the resonance frequency, and wherein the measuring coil and the high-voltage coil are wound around the same core. This produces the special advantage that the resonance frequency can be determined by means of the measuring coil, without any interference with the chamber itself being necessary.

It is preferable that the electronic control unit be adapted to measure the voltage induced in the measuring coil voltage and preferably the frequency of the voltage as well, and further preferably to determine a voltage ramp rate. Coupling the measuring coil and the high-voltage coil via the common core ensures that the frequency at the measuring coil is the same as the frequency at the high-voltage coil. When the resonance frequency is reached, the voltage induced at the measuring coil also increases to a maximum. This means it is possible, with little technical effort, to detect whether or when the resonance frequency has been reached, by monitoring the voltage curve at the measuring coil.

In another preferred embodiment of the invention, the electronic control unit has a controller comprising a first processor for determining the resonance frequency, and a driver unit comprising a second processor for driving the high-voltage source, wherein the driver unit is configured to control at least one of the following: the frequency, the pulse duration and the amplitude of the voltage of the high-voltage source. It has been found that it is advantageous to analyse the voltage induced at the measuring coil, on the one hand, and how the high-voltage source is driven, on the other hand, using two dedicated processors, because it is possible as a consequence to use small processors that require few resources. The controller is preferably adapted to transmit control commands to the driver unit, depending on the measured variables of the measuring coil, so that the frequency of the overall system approaches the resonance frequency.

According to another embodiment of the device, the high-voltage source has a primary coil which is wound around the same core. The coil referred to above as the high-voltage coil is then a secondary coil. The high-voltage source preferably has a plurality of voltage doublers which are connected in series and which are connected to the high-voltage coil.

The electronic control unit is preferably adapted to vary, automatically and in steps, at least one of the following variables at predetermined intervals: the frequency, the pulse duration and the amplitude of the voltage of the high-voltage source, preferably of the primary coil. The electronic control unit is also preferably adapted to perform, after a first variation step, a further variation step in the same direction, if the voltage induced at the measuring coil after the first variation step is higher than before, and to perform a variation step in the opposite direction if the voltage induced at the measuring coil is lower after the first variation step than before. By this means, a system is provided which adapts automatically to changing conditions in the chamber. By performing the variation steps, checks are continuously made to determine whether a higher or a lower frequency (or other parameter, such as the pulse width) at the measuring coil results in a higher induced voltage. A change is firstly made in a first direction, and if this variation results in a reduction in the voltage induced at the measuring coil, a variation is made in the opposite direction, until the variation always occurs alternatingly about a maximum. This is then the new optimal operation mode. The coil may optionally be pulsed with a frequency between 1 and 128 Hz. This preferably occurs when the optimal operation mode (the optimal resonance frequency) is reached.

The time interval from one variation step to a variation step in the opposite direction is preferably less than the interval between two variation steps in the same direction. This ensures a faster response to a change in frequency (or some other parameter such as the pulse width).

The invention also relates to a use of the device for electrically disintegrating cell clusters. The invention solves the problem it addresses by using the device with the following steps:

providing a chamber within which an electrode body is arranged,

feeding fluid containing cell clusters into the chamber,

producing an electric field in the chamber in such a way that cell clusters disintegrate,

altering the electric field by means of an electronic control unit which cooperates with a high-voltage source for the electrode body, and

determining the resonance frequency of the high-voltage source.

With regard to the advantages of such use and its preferred embodiments, reference is made to the description of the inventive device in the foregoing.

This use according to the invention is preferably developed such that determination of the resonance frequency includes:

measuring the voltage induced in a measuring coil, wherein the measuring coil and a high-voltage coil of the high-voltage source are wound around the same core.

In a preferred embodiment of the use, altering the electric field includes controlling at least one of the following:

the frequency,

the pulse duration, and

the amplitude

of the voltage of the high-voltage source.

In another preferred embodiment of the use according to the invention, this includes the step of:

varying, automatically and in steps, at least one of the following variables at predetermined intervals:

the frequency,

the pulse duration, and

the amplitude

of the voltage of the high-voltage source, preferably of the primary coil.

The use according to the invention is further developed by at least one of the steps:

performing a further variation step in the same direction if the voltage induced at the measuring coil after a first variation step is higher than before, and

performing a variation step in the opposite direction if the voltage induced at the measuring coil is lower after a first variation step than before.

The invention shall now be described in greater detail with reference to the attached Figures, in which

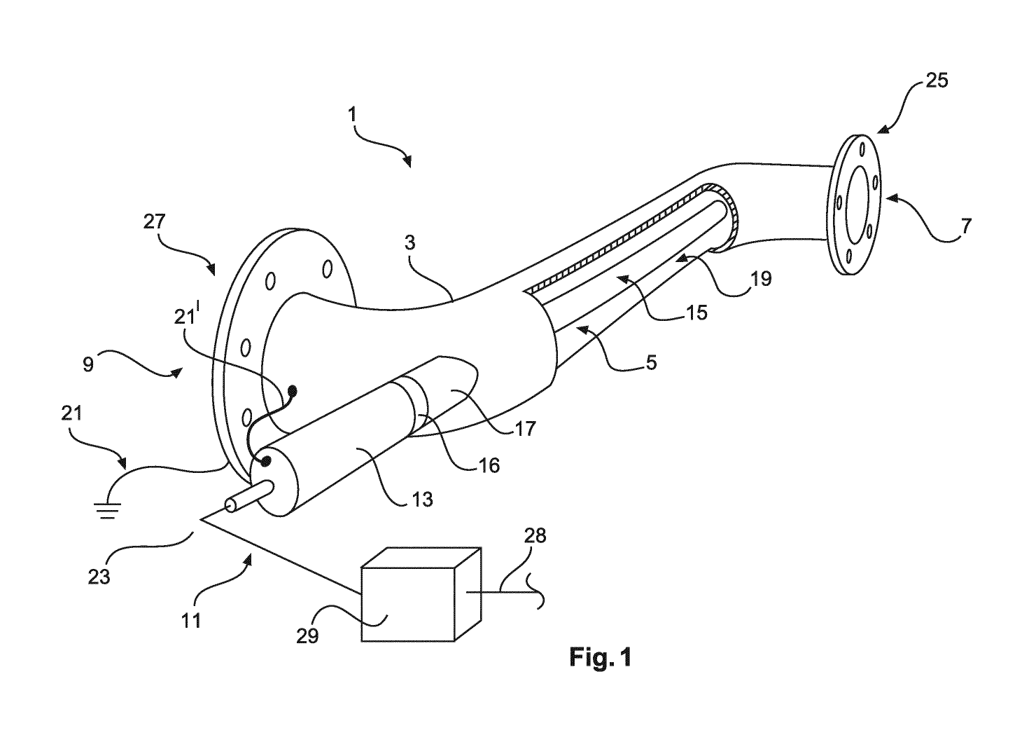

FIG. 1: shows a spatial view of the device for disintegrating cell clusters according to the invention,

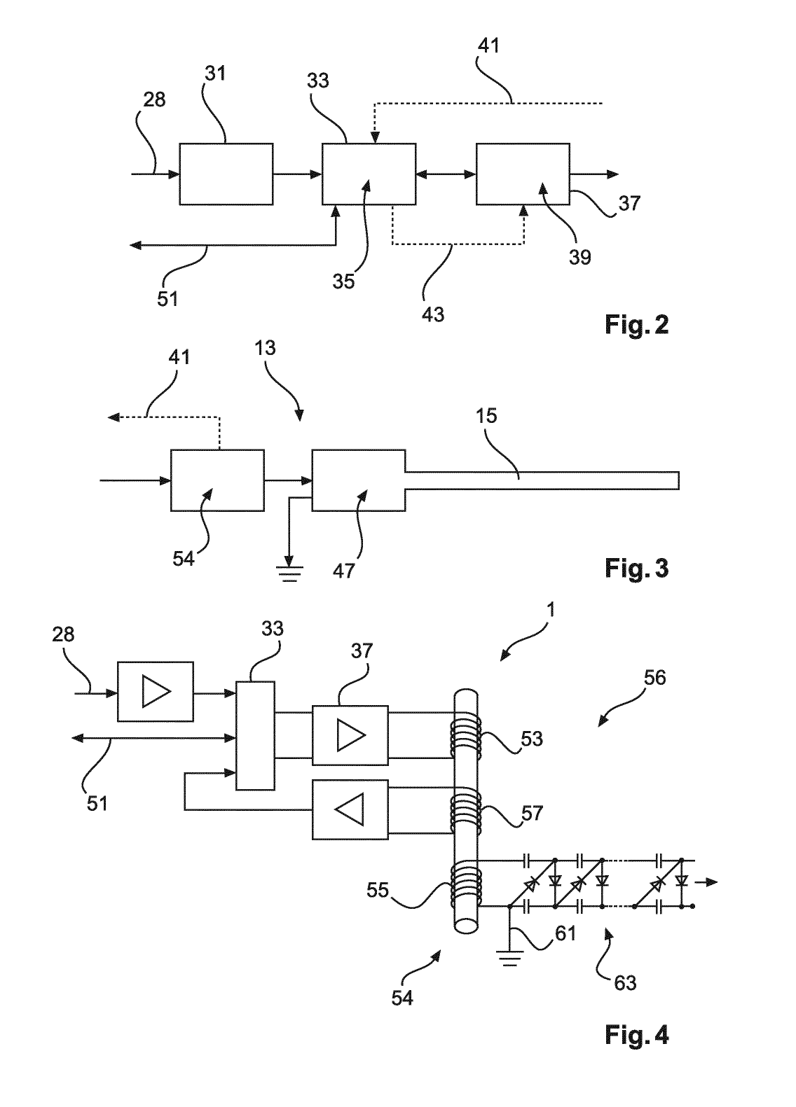

FIG. 2 shows a schematic partial view of the functional structure of the device according to the invention,

FIG. 3 shows another schematic partial view of the functional functional structure of the device according to the invention, and

FIG. 4 shows yet another schematic partial view of the functional structure of the device according to the invention.

Device 1 shown in FIG. 1 has a housing 3. Sections of housing 3 are cylindrical in shape. A chamber 5, sections of which are in the shape of a hollow cylinder, is arranged inside housing 3. An inlet 7 for receiving fluid into chamber 5 and an outlet 9 for discharging fluid from chamber 5 are arranged at two opposite ends of housing 3. Device 1 has an electrode unit 11. Electrode unit 11 has an electrode head 13 and an electrode body 15. By means of an electrode guide 17 which is encircled by housing 3, electrode unit 11 is received in such a way that electrode body 15 extends inside chamber 5 of housing 3. Electrode guide 17 is in the form of a tubular extension and defines a central opening 16. Electrode body 15 is preferably connected by means of a screw connection (not shown) to housing 3 and electrode guide 17. As an option, electrode body 15 is mounted on a side of housing 3 opposite electrode guide 17 with another electrode guide (not shown). Chamber 5 has a wall 19, which is electrically insulated from electrode body 15. As an option, wall 19 of chamber 5 is electrically insulated in sections, or clated with a dielectric material. Housing 3 and electrode unit 11 are earthed by means of an earthing 21. As an option, housing 3 and electrode head 13 are likewise connected by means of an earthing 21′.

Inlet 7 has a flange 25 for connecting to a piping system or for connecting to a further, adjacent device 1 (not shown). Outlet 9 has a flange 27 which is likewise designed for connecting to a piping system or for connecting to an adjacent device 1. A disintegrating device according to the present invention is formed by a single device 1 or by joining a plurality of devices 1 by means of flanges 25, 27.

Electrode unit 11 is designed to produce an electric field between electrode body 15 and wall 19 of chamber 5. Device 1 has an electronic control unit 29 for driving electrode unit 11. This is shown in more detail in FIG. 2.

Electronic control unit 29 has a power unit 31 which includes a voltage input 28 for connection to a 230V, 50 Hz AC voltage source, for example. The power unit is connected to a controller 33 containing a first processor 35. Controller 33 is adapted to determine the resonance frequency of the system comprising the high-voltage source and the chamber/electrode body.

Controller 33 is connected by signal line 43 to a driver unit 37 which contains a second processor 39 and which is adapted to drive a coil unit 54, which is part of high-voltage source 56 (see FIG. 4). A data exchange line 51 is provided for fetching data from the controller and/or for programming or controlling the latter.

In the region of electrode head 13 (FIG. 3), a high-voltage coil 47 is provided which results in the voltage fed to coil unit 54 being multiplied. The voltage provide by high-voltage coil 47 is likewise applied between electrode body 15 and wall 19. Controller 33 is also connected by means of a signal line 41 to high-voltage source 56, so as to be able to determine the resonance frequency of the latter. FIG. 4 illustrates how this can be advantageously implemented.

Coil unit 54 shown in FIG. 4 has a primary coil 53 which is connected to driver unit 37 and which has a first number of windings. Coil unit 54 also has a secondary coil 55 having a second number of windings, preferably a multiple of the first number of windings in the primary coil. Finally, coil unit 54 has a measuring coil 57. All the coils 53, 55, 57 are wound around the same coil core, for example a ferrite core. The input voltage is transformed by means of primary and secondary coils 53, 55. The voltage induced in measuring coil 57, and preferably other variables such as the frequency, are measured by the controller or are measured and transmitted to the latter.

The secondary coil is coupled to a plurality of voltage doublers 63 and earthed by means of line 61. The multiplied voltage is applied between electrode body 15 and wall 19.

1-9. (canceled)

10. A device for electrically disintegrating cell clusters, comprising:
an electrode unit which has an electrode head and an electrode body;
a chamber within which the electrode body is arranged, wherein the chamber has a wall which is electrically conductive in some sections or completely and which is electrically insulated from the electrode body, and wherein the chamber has an inlet for receiving fluid containing cell clusters;
a high-voltage source which is arranged in the electrode head and which is designed to produce an electric field by applying a voltage between the electrode body and the wall; and
an electronic control unit which interacts with the high-voltage source in order to change the electric field,
wherein the electronic control unit has means for determining the resonance frequency of the high-voltage source,
wherein the high-voltage source has a primary coil which is wound around the same core, and the high-voltage coil is a secondary coil.

11. The device according to claim 10, wherein the high-voltage source has a high-voltage coil and a measuring coil, wherein the measuring coil is connected to the means for determining the resonance frequency, and wherein the measuring coil and the high-voltage coil are wound around the same core.

12. The device according to claim 11, wherein the electronic control unit is adapted to measure the voltage induced in the measuring coil and preferably the frequency of the voltage as well, and further preferably to determine a voltage ramp rate.

13. The device according to claim 10, wherein the electronic control unit has a controller comprising a first processor for determining the resonance frequency, and a driver unit comprising a second processor for driving the high-voltage source, wherein the driver unit is configured to control at least one of the following:
the frequency,
the pulse duration, and
the amplitude
of the voltage of the high-voltage source.

14. The device according to claim 10, wherein the high-voltage source has a plurality of voltage doublers which are connected in series and which are connected to the high-voltage coil.

15. The device according to claim 10, wherein the electronic control unit is adapted to vary, automatically and in steps, at least one of the following variables at predetermined intervals:
the frequency,
the pulse duration, and
the amplitude
of the voltage of the high-voltage source, preferably of the primary coil.

16. The device according to claim 15, wherein the electronic control unit is adapted, after completing a variation step:
to perform a further variation step in the same direction if the voltage induced at the measuring coil after the variation step is higher than before; and
to perform a variation step in the opposite direction if the voltage induced at the measuring coil is lower after the variation step than before.

17. The device according to claim 16, wherein the interval from one variation step to a variation step in the opposite direction is less than the interval between two variation steps in the same direction.
